(19) United States
(12) Patent Application Publication
(10) Pub. No.: US 2021/0082571 A1
(43) Pub. Date: Mar. 18, 2021

(54) SYSTEM AND METHOD FOR MANAGING AND COORDINATING SURGICAL SCHEDULING INVOLVING SURGEONS, PATIENTS, ANESTHESIOLOGISTS, MEDICAL FACILITIES AND/OR SURGICAL IMPLANT AND INSTRUMENTATION VENDORS

(71) Applicants: Bryan Larsen, Las Vegas, NV (US); George Gluck, Las Vegas, NV (US); Jeff Sorenson, Henderson, NV (US)

(72) Inventors: Bryan Larsen, Las Vegas, NV (US); George Gluck, Las Vegas, NV (US); Jeff Sorenson, Henderson, NV (US)

(21) Appl. No.: 16/573,949

(22) Filed: Sep. 17, 2019

(51) Int. Cl. G16H 40/20; G06Q 10/08; G06Q 10/10; G06Q 30/02; H04W 4/029

(52) U.S. Cl. CPC G16H 40/20; G06Q 10/0833; H04W 4/029; G06Q 30/0282; G06Q 10/1093

(57) ABSTRACT

A computer-implemented system comprising: memory and a hardware processor configured to: generate a dynamic, digital community calendar accessible by at least registered surgeons and one or more registered implant vendors via one or more computer devices; provide bi-directional electronic communications between registered surgeons and one or more registered implant vendors; provide surgeons with detailed digital information regarding implant inventories of one or more registered implant vendors; provide surgeons with a digital order interface configured to allow surgeons to order implants from one or more vendors, the order including a time, date and location for delivery of the order; provide the one or more vendors with a digital acceptance interface configured to allow the one or more vendors to accept the order; provide real-time status of order delivery; and facilitate payment for the order.

### FIELD OF THE INVENTION

The embodiments of the present invention relate to a system and method for coordinating surgical schedules for all interested parties.

### BACKGROUND

A surgical procedure requiring the use of an implant requires at least a surgeon, patient, anesthesiologist, medical facility and implant vendor. Given all of the moving parts, it can be challenging to coordinate the schedules of all parties for the surgical procedure. This is especially true when the implant vendor is involved.

It would be advantageous to develop an easy-to-use system and method for coordinating surgical schedules for all interested parties including implant vendors. Preferably, the system and method are facilitated by computer devices including mobile smart devices.

### SUMMARY

One embodiment of the present invention is directed to a computer-implemented system comprising: memory and a hardware processor configured to: generate a dynamic, digital community calendar accessible by at least registered surgeons and one or more registered implant vendors via one or more computer devices; provide bi-directional electronic communications between said registered surgeons and said one or more registered implant vendors; provide said surgeons with detailed digital information regarding implant inventories of said one or more registered implant vendors; provide said surgeons with a digital order interface configured to allow surgeons to order implants from said one or more vendors, said order including a time, date and location for delivery of said order; provide said one or more vendors with a digital acceptance interface configured to allow said one or more vendors to accept said order; provide real-time status of order delivery; and facilitate payment for said order.

The vendor—surgeon relationship is important in the surgical space. Surgeons needs to be able to find reliable implants while vendors desire to sell their implants. Moreover, once ordered, the delivery timing of the implants is crucial to the underlying surgery. The computer-implemented system and method detailed herein provide a dynamic, easy-to-use ordering and scheduling platform for surgical procedures involving third party vendors.

Another embodiment of the present invention is directed to a computer-implemented method comprising: generating, via at least a hardware processor and memory, a dynamic, digital community calendar accessible by at least registered surgeons and one or more registered implant vendors via one or more computer devices; providing, via at least a hardware processor and memory, bi-directional electronic communications between said registered surgeons and said one or more registered implant vendors; providing, via at least a hardware processor and memory, said surgeons with detailed digital information regarding implant inventories of said one or more registered implant vendors; providing, via at least a hardware processor and memory, said surgeons with a digital order interface configured to allow surgeons to order implants from said one or more vendors, said order including a time, date and location for delivery of said order; providing, via at least a hardware processor and memory, said one or more vendors with a digital acceptance interface configured to allow said one or more vendors to accept said order; providing, via at least a hardware processor and memory, real-time status of order delivery; and facilitating, via at least a hardware processor and memory, payment for said order.

Other variations, embodiments and features of the present invention will become evident from the following detailed description, drawings and claims.

### BRIEF DESCRIPTION OF THE DRAWINGS

FIG. 1 illustrates a block diagram of a wireless network of the type that may facilitate the embodiments of the present invention;

FIG. 2 illustrates a first user capabilities diagram according to the embodiments of the present invention;

FIG. 3 illustrates a second user capabilities diagram according to the embodiments of the present invention;

FIG. 4 illustrates a third user capabilities diagram according to the embodiments of the present invention;

FIG. 5 illustrates a fourth user capabilities diagram according to the embodiments of the present invention;

FIG. 6 illustrates a fifth user capabilities diagram according to the embodiments of the present invention;

FIGS. 7A-7B illustrate a dashboard interface according to the embodiments of the present invention;

FIG. 8 illustrates an order model according to the embodiments of the present invention;

FIG. 9 illustrates a relationship model involving surgeons, medical facilities and assistants according to the embodiments of the present invention;

FIG. 10 illustrates a relationship model involving vendors and products according to the embodiments of the present invention;

FIG. 11 illustrates an orders screen shot according to the embodiments of the present invention;

FIG. 12 illustrates a sales rep screen shot according to the embodiments of the present invention;

FIG. 13 illustrates a products screen shot according to the embodiments of the present invention;

FIG. 14 illustrates a doctors' screen shot according to the embodiments of the present invention;

FIGS. 15A-15X illustrate exemplary screen shots according to the embodiments of the present invention;

FIG. 16 illustrates a flow chart detailing a method of utilizing the system described herein by doctors and doctors' assistants according to the embodiments of the present invention; and

FIG. 17 illustrates a flow chart detailing a method of utilizing the system described herein by vendors and vendor/sales rep according to the embodiments of the present invention.

### DETAILED DESCRIPTION

For the purposes of promoting an understanding of the principles in accordance with the embodiments of the present invention, reference will now be made to the embodiments illustrated in the drawings and specific language will be used to describe the same. It will nevertheless be understood that no limitation of the scope of the invention is thereby intended. Any alterations and further modifications of the inventive feature illustrated herein, and any additional applications of the principles of the invention as illustrated herein, which would normally occur to one skilled in the relevant art and having possession of this disclosure, are to be considered within the scope of the invention claimed.

Those skilled in the art will recognize that the embodiments of the present invention involve both hardware and software elements which portions are described below in such detail required to construct and operate a system and method for coordinating surgical schedules for surgeons, medical facilities, vendors, patients, assistants, anesthesiologists and others involved in a given surgical procedure utilizing medical implants.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.), or an embodiment combining software and hardware. Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), and optical storage device, a magnetic storage device, or any suitable combination of the foregoing. In the context of this document, a computer readable storage medium may be any tangible medium that can contain or store a program for use by or in connection with an instruction execution system, apparatus, or device. Cloud storage may be used as well.

A computer readable signal medium may include a propagated data signal with computer readable program code embodied thereon, for example, in baseband or as part of a carrier wave. Such a propagated signal may take any variety of forms, including, but not limited to, electromagnetic, optical, or any suitable combination thereof. A computer readable signal medium may be any computer readable medium that is not a computer readable storage medium and that can communicate, propagate, or transport a program for use by or in conjunction with an instruction execution system, apparatus, or device.

Program code embodied on a computer readable medium may be transmitted using any appropriate medium, including but not limited to wireless, wireline, optical fiber cable, RF and the like, or any suitable combination of the foregoing.

Computer program code for carrying out operations for aspects of the present invention may be written in any combination of one or more programming languages, including an object-oriented programming language such as Java, Smalltalk, C++ or the like or conventional procedural programming languages, such as the “C” programming language, AJAX, PHP, HTML, XHTML, Ruby, CSS, Python, GO or similar programming languages. The programming code may be configured in an application, an operating system, as part of a system firmware, or any suitable combination thereof. The programming code may execute entirely on the user's computer, partly on the user's computer, as a standalone software package, partly on the user's computer and partly on a remote computer or entirely on a remote computer or server as in a client/server relationship sometimes known as cloud computing. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special-purpose computer, or other programmable data processing apparatus to produce a machine, such that the instructions, which execute via the processor of the computer or other programmable data processing apparatus, create means for implementing the functions/acts specified in the flowchart and/or block diagram.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram.

The computer program instructions may also be loaded onto a computer, other programmable data processing apparatus, or other devices to cause a series of operational steps to be performed on the computer, other programmable apparatus or other devices to produce a computer-implemented process such that the instructions which execute on the computer or other programmable apparatus provide processes for implementing the functions/acts specified in the flowchart and/or block diagrams. As used herein, a “terminal” should be understood to be any one of a general purpose computer, as for example a personal computer or a laptop computer, a client computer configured for interaction with a server, a special purpose computer such as a server, or a smart phone, soft phone, tablet computer, personal digital assistant or any other machine adapted for executing programmable instructions in accordance with the description thereof set forth above.

The doctor-vendor relationship and ability to coordinate with one another is critical in the surgery space. When surgical implants or specialized equipment are required and requested, it is critical that such items be delivered to the medical facility by the vendor at least 24 hours to allow for sterilization. Any break in this schedule delays surgery risking patient safety and causing stress to both the patient and surgeon. The system and method detailed herein seek to ensure surgical scheduling and related activities run smoothly for all interested parties.

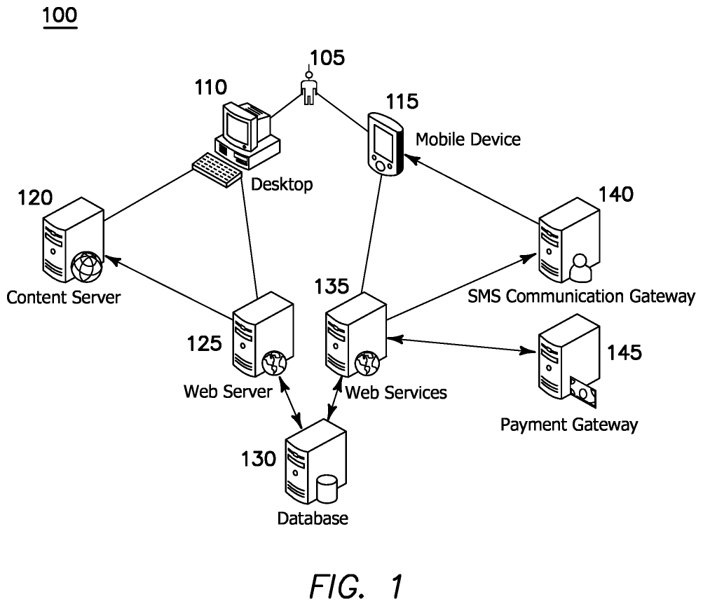

FIG. 1 shows an exemplary system architecture 100 according to the embodiments of the present invention. As shown, a user 105 may access the system via a desk top computer 110 or smart mobile device 115. The system and method may be web-based and/or app-based. The desktop computer 110 interfaces with a content server 120 which interfaces with a web-server 125 which communicates with or manages a database 130. The database 130 and web-server 125 may be a single piece of equipment. Smart mobile devices 115 access the database 130 via web-services 135. The smart mobile devices 115 may also interface with a SMS Communication Gateway 140 facilitating messaging between different users. A Payment Gateway 145 provides a payment solution for surgeons. Those skilled in the art will recognize that other system architectures may facilitate the embodiments of the present invention.

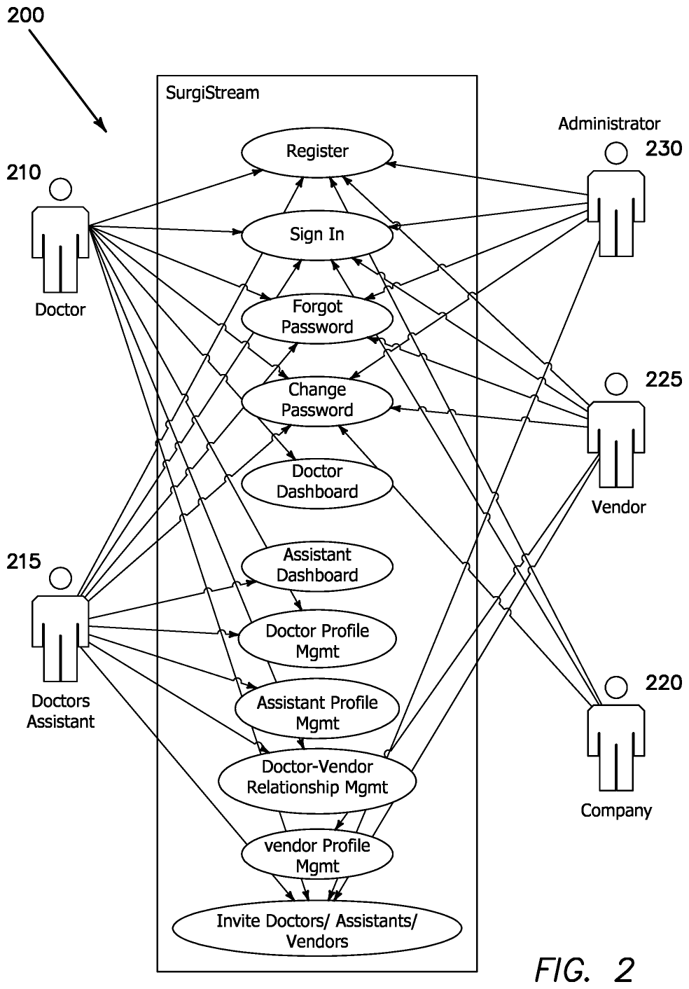

FIGS. 2 through 6 detail how various users interact with the system. As described herein, the users comprise: patients, surgeons, surgeons' assistants, medical device companies, sales representatives for medical device companies (aka vendors) and administrators. In other embodiments, other users (e.g., patient's family members) may be permitted to utilize the system.

FIG. 2 is a first diagram 200 of a series of functions/features of the system related to user management comprising registration, signing-in, forgetting password, changing password, doctor dashboard, assistant dashboard, doctor profile management, assistant profile management, doctor-vendor relationship management, vendor profile management and invite doctors/assistants/vendors. Access to the functions/features is selectively distributed to the users based on need. As shown, the users comprise doctors 210, doctors' assistants 215, medical device companies 220, vendors 225 and administrators 230.

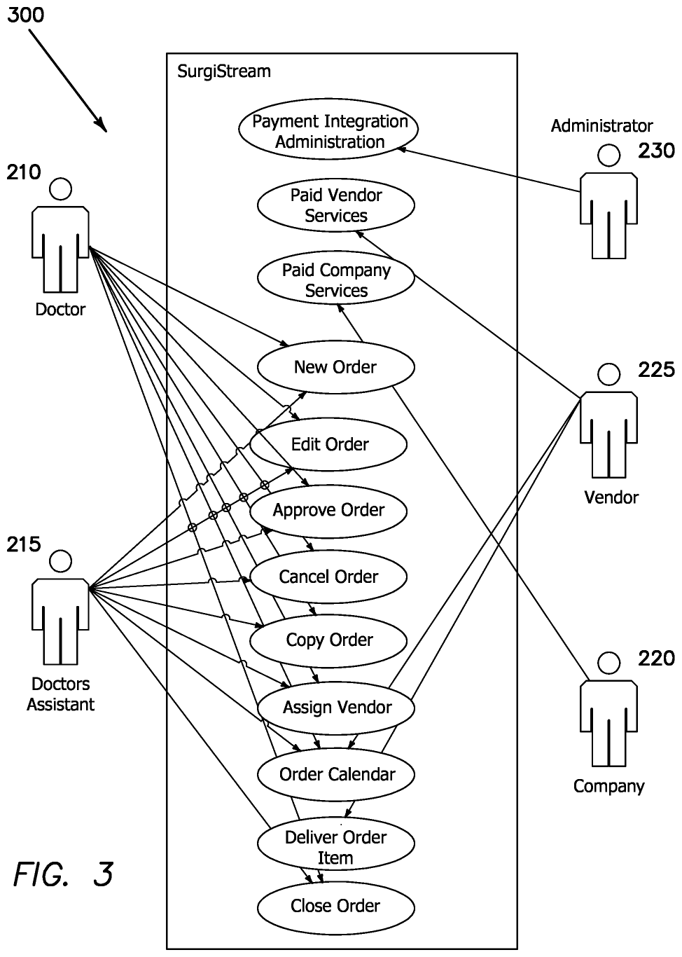

FIG. 3 is a second diagram 300 of a series of functions/features of the system related to orders comprising payment integration management, paid vendor services, paid company services, new order, edit order, approve order, cancel order, copy order, assign vendor, order calendar, deliver order and close order. Access to the functions/features is selectively distributed to the users based on need. As shown, the users comprise doctors 210, doctors' assistants 215, medical device companies 220, vendors 225 and administrators 230.

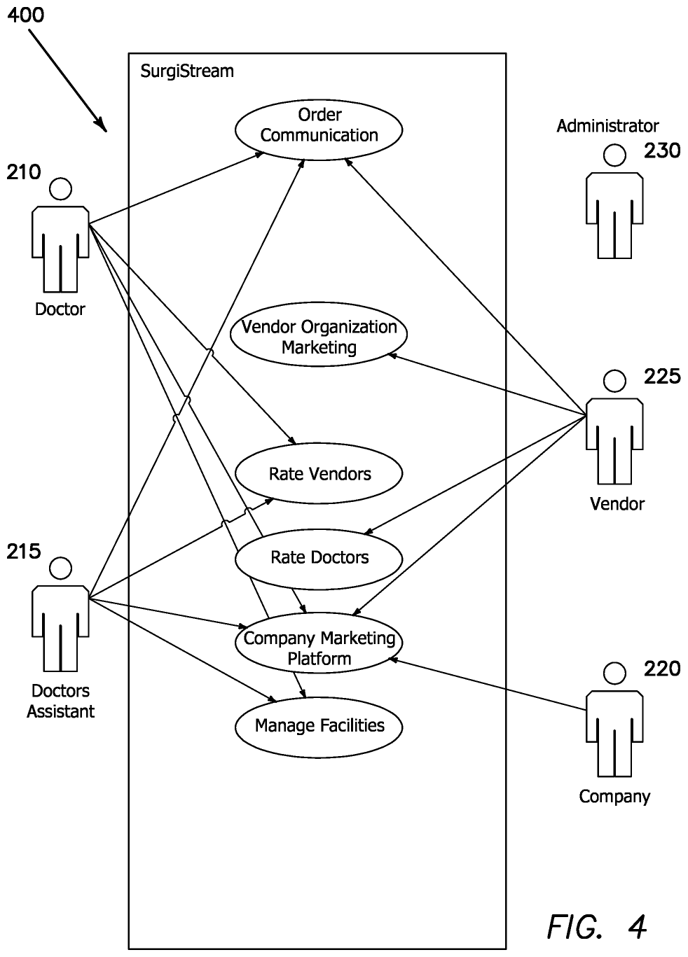

FIG. 4 is a third diagram 400 of a series of miscellaneous functions/features of the system comprising order communication, vendor marketing, vendor rating, doctor rating, company marketing platform and medical facilities management. Access to the functions/features is selectively distributed to the users based on need. As shown, the users comprise doctors 210, doctors' assistants 215, medical device companies 220, vendors 225 and administrators 230.

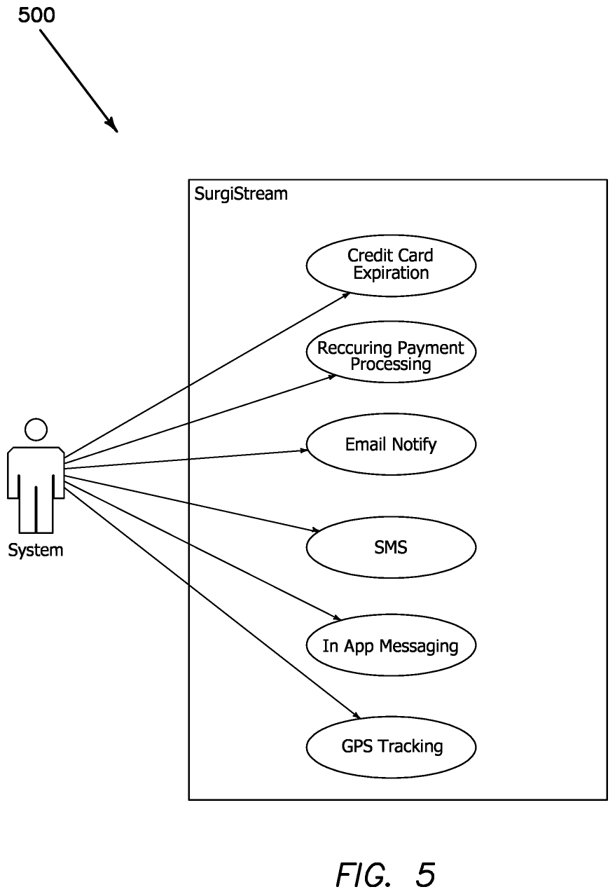

FIG. 5 is a fourth diagram 500 of a series of system capabilities comprising credit card expiration, recurring payment processing, email notification, SMS messaging, in-app messaging and GPS tracking.

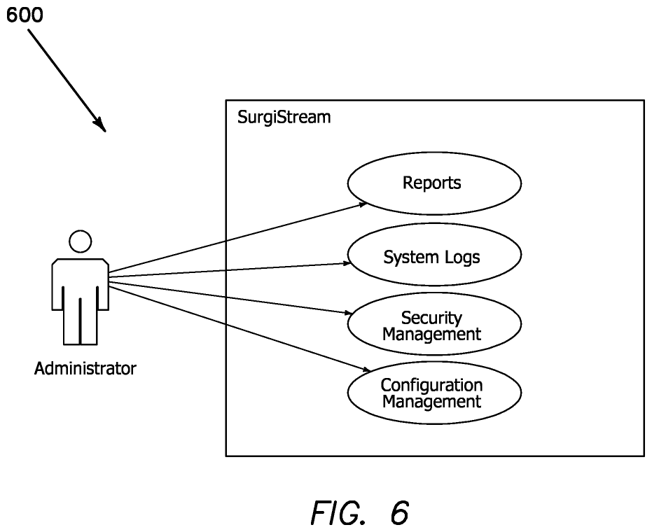

FIG. 6 is a fifth diagram 550 of a series of administrator capabilities comprising reports, system logs, security management and configuration management.

Access to the system 100 first requires registration by at least doctors, doctors' assistants and vendors. The registration process comprises entering personal information and a password or similar security protocol which may be stored in database 130. Once registered, the users have access to the functionality and features as shown in FIGS. 2-4. The users may initially enter profiles into the system 100 via a user interface. A doctor's and doctor's assistant's profile may include, among other things, schooling history, employment history, medical expertise, certificates earned, associated medical facilities/doctors/assistants and the like. A vendor's profile may include, among other things, employment history, implant expertise and associated medical manufacturers and the like.

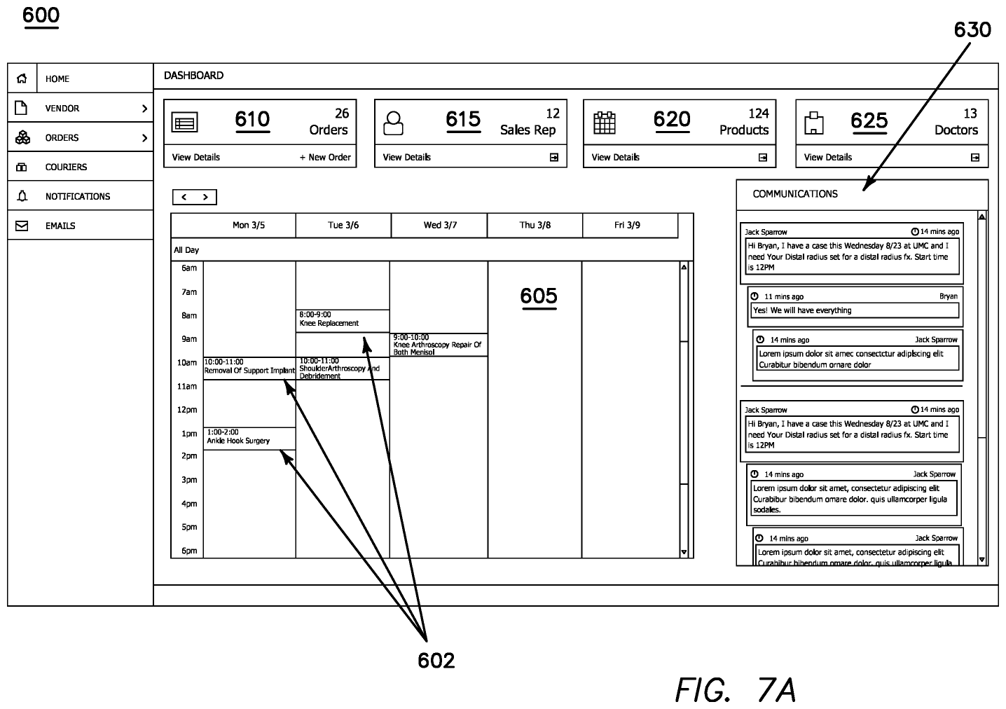

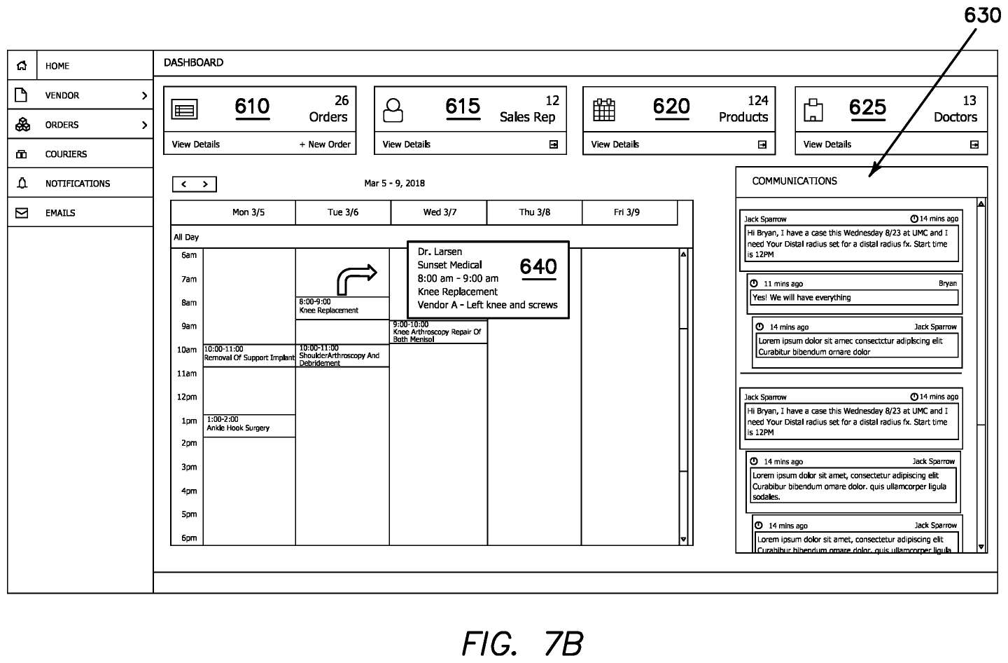

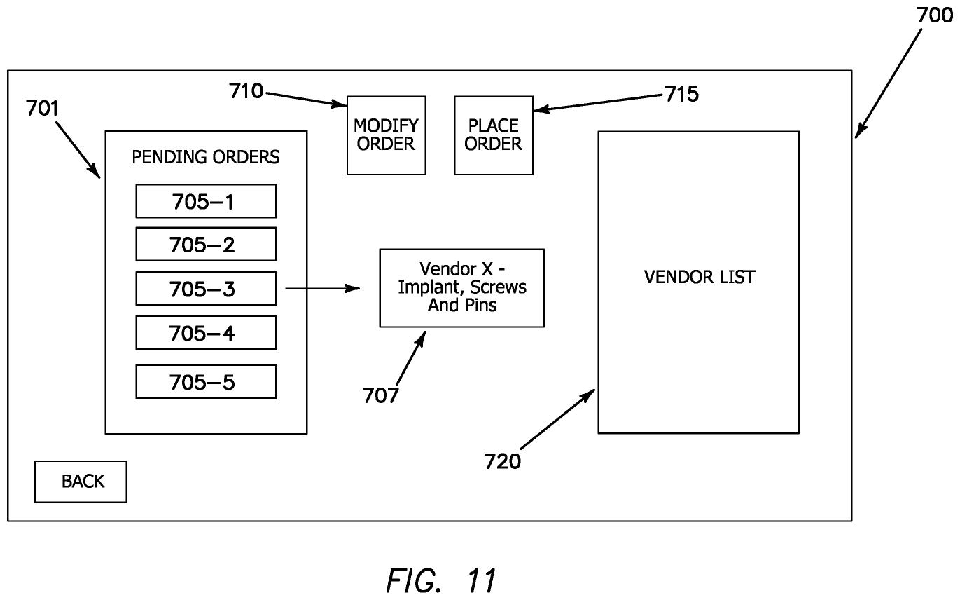

One primary function of the system 100, namely the dynamic calendar, is facilitated by a dashboard interface 600 shown in FIG. 7A. The home page of the dashboard interface 600 includes a dynamic calendar 605, orders link 610, sales reps/vendors link 615, products/companies link 620 and doctors/assistants link 625. The user may click on any of the links 610-625 to pull up web or app pages dedicated to each subject as shown in more detail in FIGS. 11-14. The dynamic calendar 605 provides surgery dates, surgery times and surgery types. In one embodiment, each calendar entry 602 is color-coded or otherwise distinguishable based on the doctor (e.g., Doctor Smith=Red; Doctor Jones=Blue, Doctor Gluck=Green). In another embodiment, each entry is color-coded or otherwise distinguishable based on the vendor. In one embodiment, the color-coded entries are based on the user viewing the dynamic calendar. In other words, a doctor viewing the dynamic calendar 605 views color-coded entries based on doctors whereas a vendor viewing the dynamic calendar 605 views color-coded entries based on vendors (e.g., Vendor A=Red; Vendor B=Blue, Vendor C=Green). Clicking on any calendar entry 602 pulls up a details box 640, including the doctor's name, procedure, procedure location, procedure time, vendor name and products, as shown in FIG. 7B. The dashboard interface 600 further includes a substantially real-time messaging or communications window 630 permitting various users to communicate with one another in substantially real time.

Order link 610 permits a user to advance to an order webpage 700 as shown in FIG. 11. FIG. 11 shows an exemplary order webpage 700. The order webpage 700 includes a list 701 of ordered items 705-1 through 705-5, a modify order link 710, place order link 715 and vendor list 720. Passing the cursor over an ordered item (705-3 in this instance) causes a larger window 707 to open with order details.

In one embodiment, doctors and doctor's assistants may place orders. If the doctor's assistant enters the order, the assistant selects the doctor from a list of doctors for which the order has been placed. The assistant also selects the medical facility from a list of medical facilities to where the order should be delivered. The order may include related information including (i) body part to be operated on; (ii) surgical procedure; (iii) patient details; (iv) date and time of surgery; (v) surgery sequence number and (vi) date and time the order needs to be delivered. Once placed, the vendor, doctor and doctor's assistant receive notification via SMS, push notice and/or email.

An order that is initially created is designated as new and accessible only to the doctor or doctor's assistant. While designated as new, the order may be modified. Once the order is approved, any modifications to the order are immediately transmitted (e.g., SMS and email) to the subject vendor. In one embodiment, once an order is approved, a sales or vendor rep is assigned to the order. Alternatively, sales or vendor reps may select orders as they are submitted. Any change to the surgery schedule or cancellation entered into the system by the doctor or doctor's assistant are immediately transmitted (e.g., SMS and email) to the sales rep and vendor for the appropriate response.

Tracking orders begin when the implants and/or equipment are picked up by the courier. A mapping function permits the doctor and doctor's assistant to track the status of the delivery. The mapping function may be part of the system or may be a third-party application utilized by the courier. The order may also be geolocated to ensure the order was delivered to the proper facility.

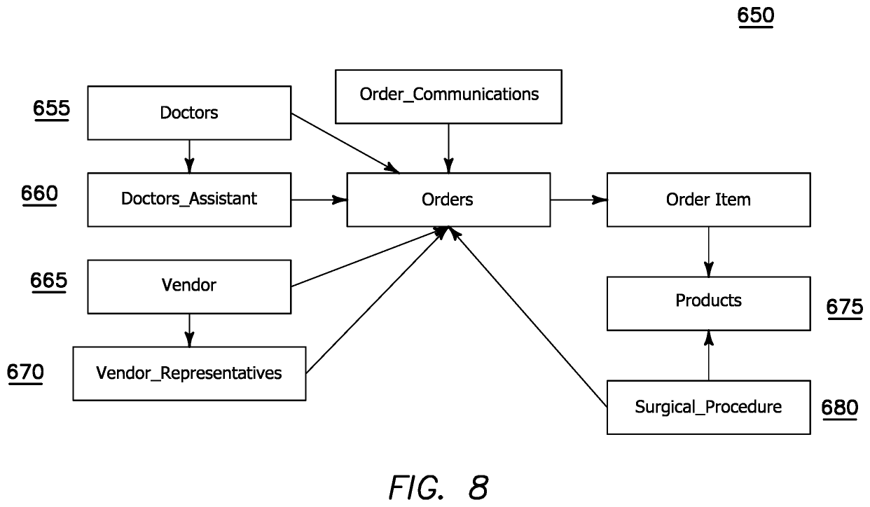

FIG. 8 shows an order model 650 according to the embodiments of the present invention. The model 650 depicts that the doctor 655 and doctor's assistant 660 can place orders while the vendor 665 and vendor/sales reps 670 can monitor and fulfill orders. As shown, products 675 and surgical procedures 680 are part of the ordering process.

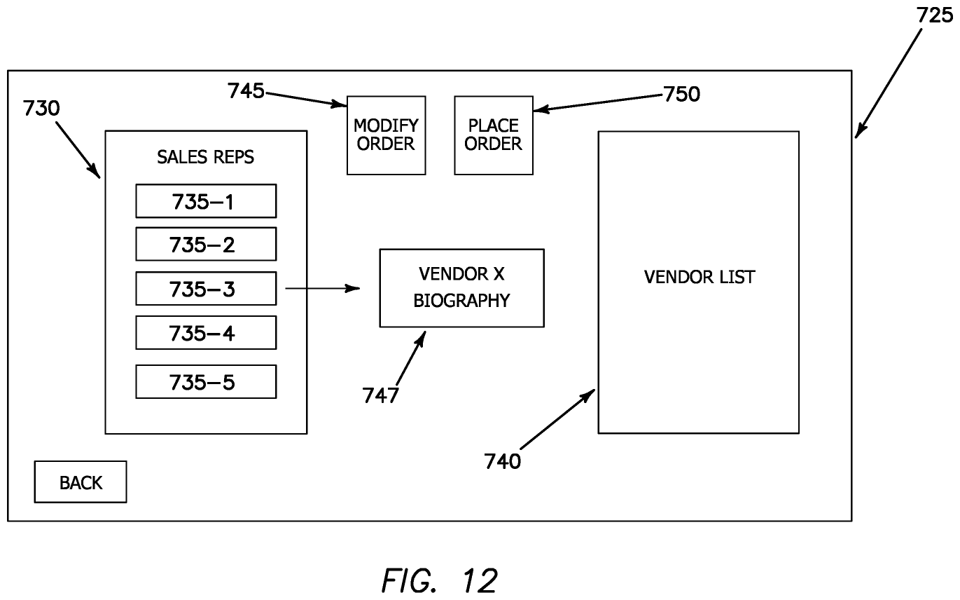

Sales Rep link 615 permits a user to advance to a sales rep webpage 725 as shown in FIG. 12. FIG. 12 shows an exemplary sales rep webpage 725. The sales rep webpage 725 includes a list 730 of sales reps 735-1 through 755-5, a vendor list 740 modify order link 745 and place order link 750. Passing the cursor over a sales rep (735-3 in this instance) causes a larger window 747 to open with sales rep details, including the vendor he or she works with and a biography.

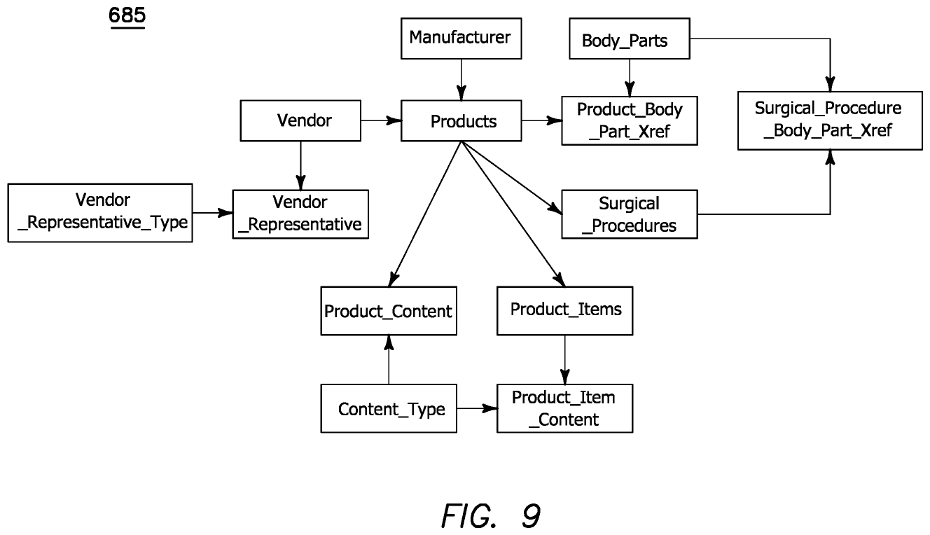

FIG. 9 shows a vendor/products relationship model 685 according to the embodiments of the present invention.

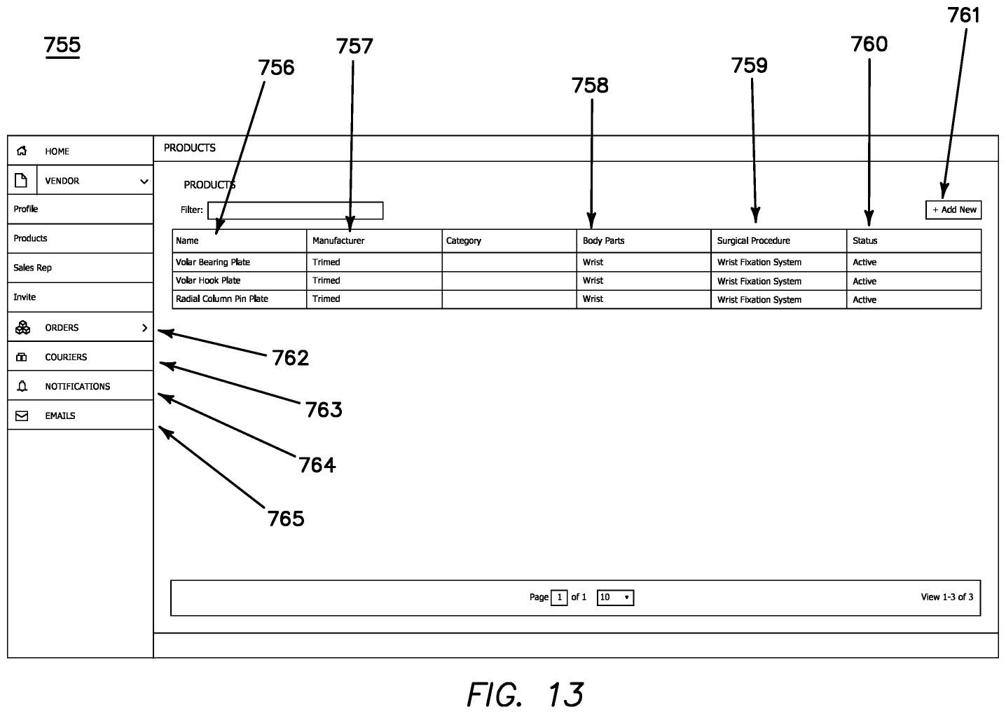

Products link 620 permits a user to advance to a products webpage 755 as shown in FIG. 13. FIG. 13 shows an exemplary products webpage 755 based on a particular vendor. The products webpage 755 includes a list of products 756, manufacturer/vendor 757, body part 758 on which the product is to be used, surgical procedure 759 for which the product is to be used and status 760. An add a new product link 761 permits the user to add a product. The exemplary products webpage 755 also includes links to orders 762, couriers 763, notifications 764 and emails 765.

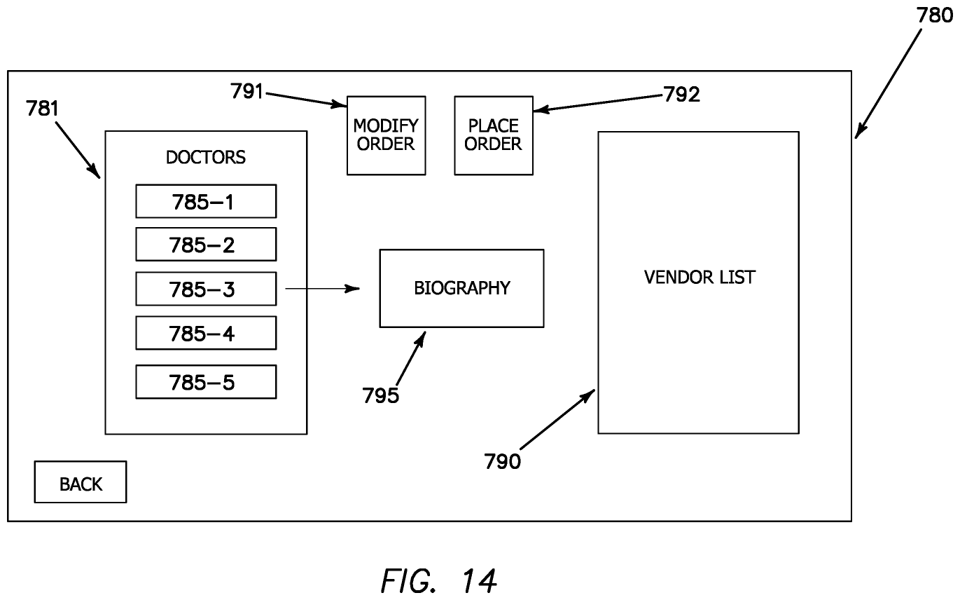

Doctors' link 770 permits a user to advance to a doctors' webpage 780 as shown in FIG. 14. FIG. 14 shows an exemplary doctors' webpage 780. The doctors' webpage 780 includes a list 781 of doctors 785-1 through 785-5, a vendor list 790, modify order link 791 and place order link 792.

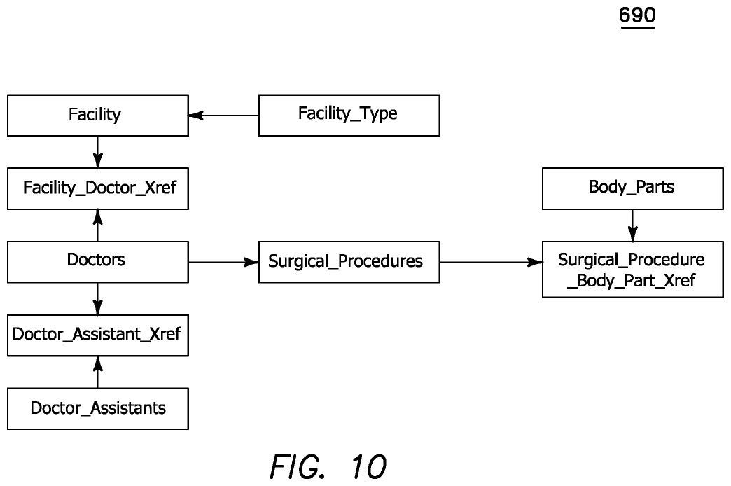

FIG. 10 shows a doctors/facility/doctors' assistant relationship model 690 according to the embodiments of the present invention.

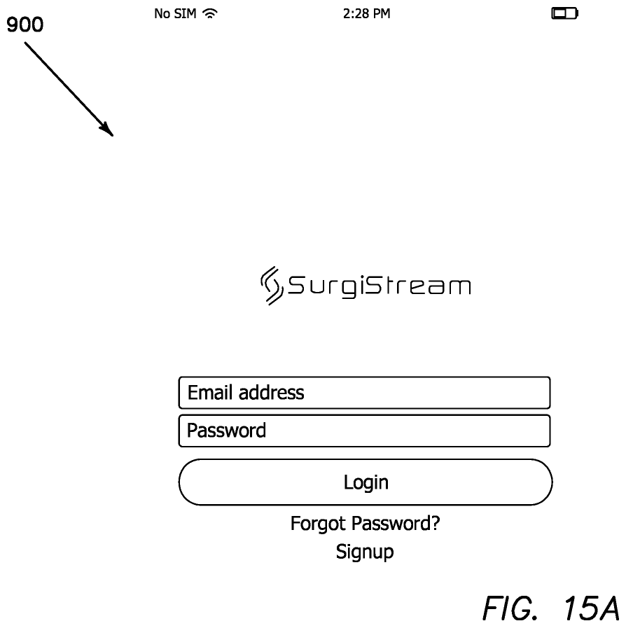

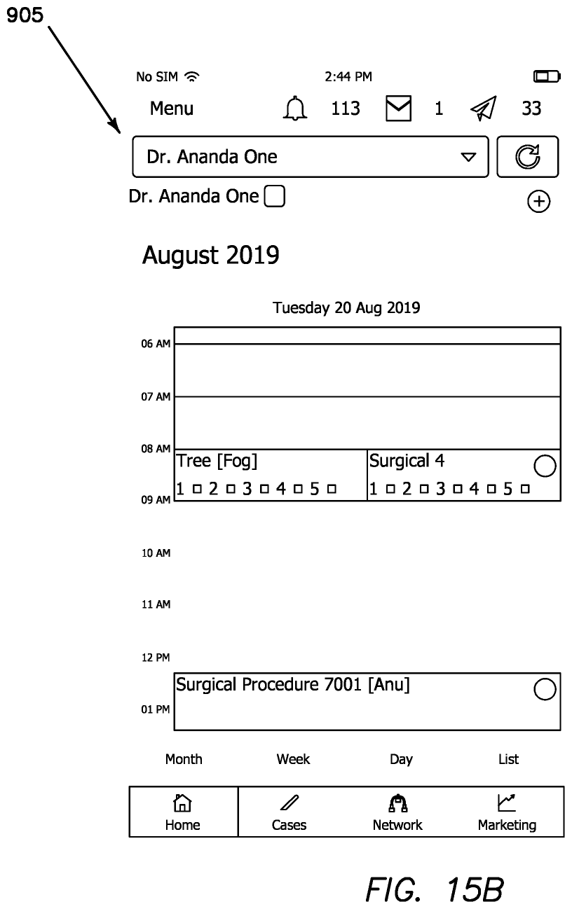

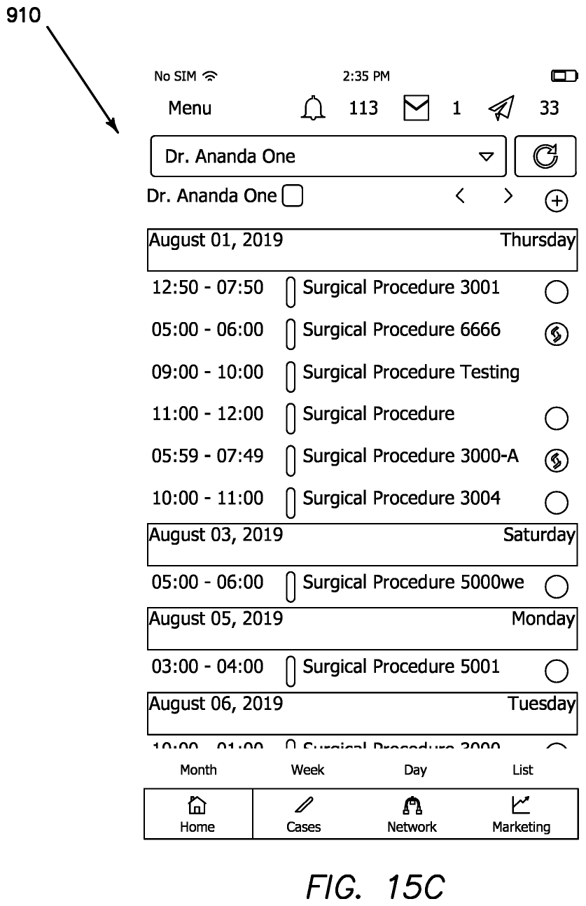

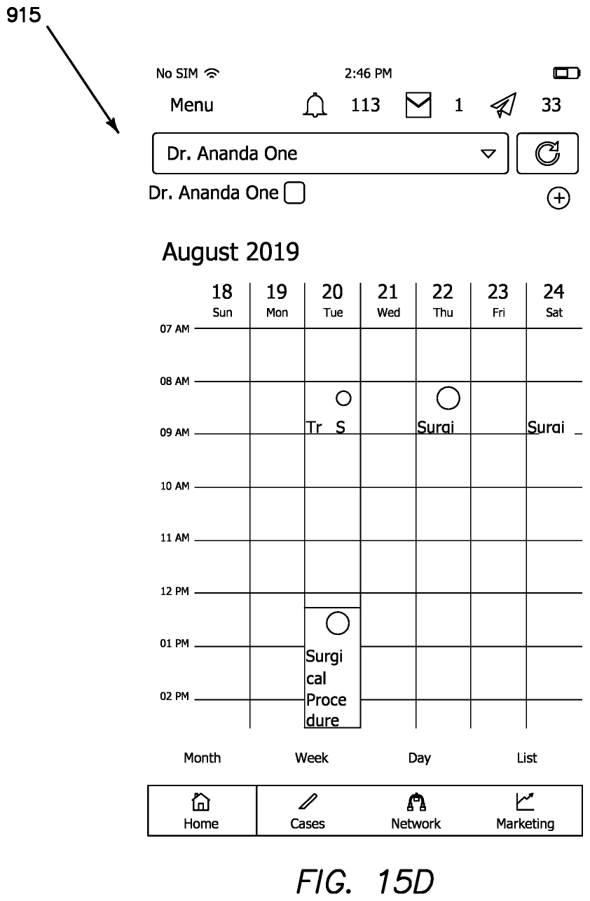

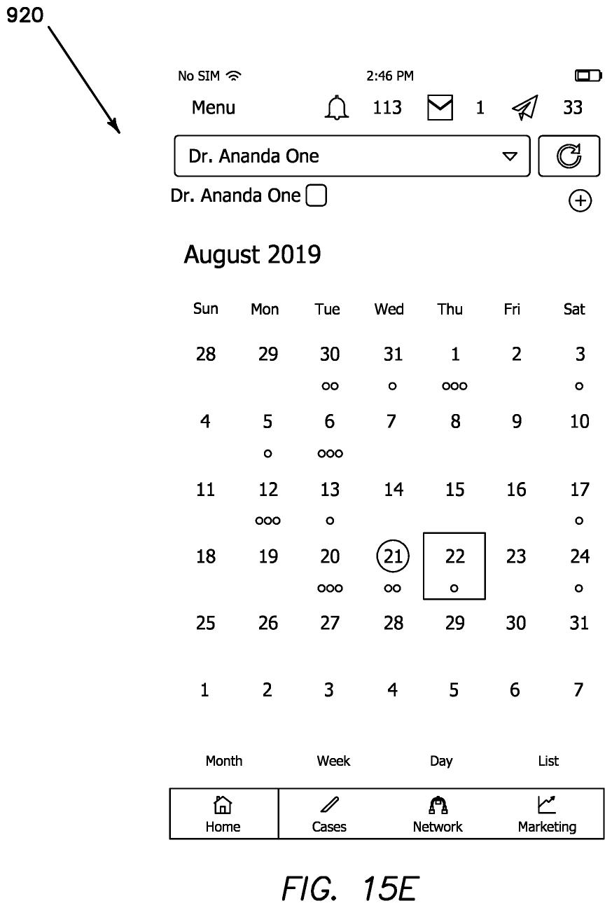

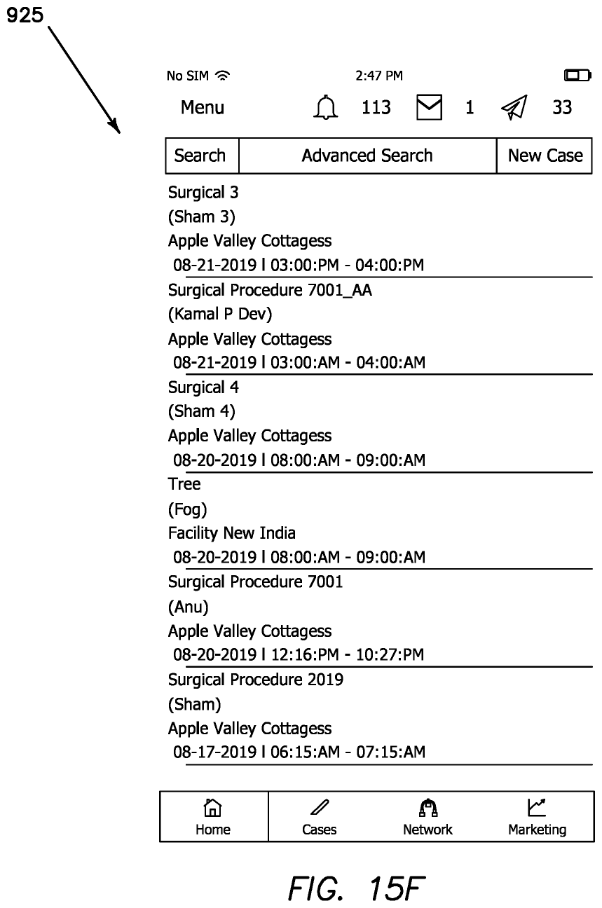

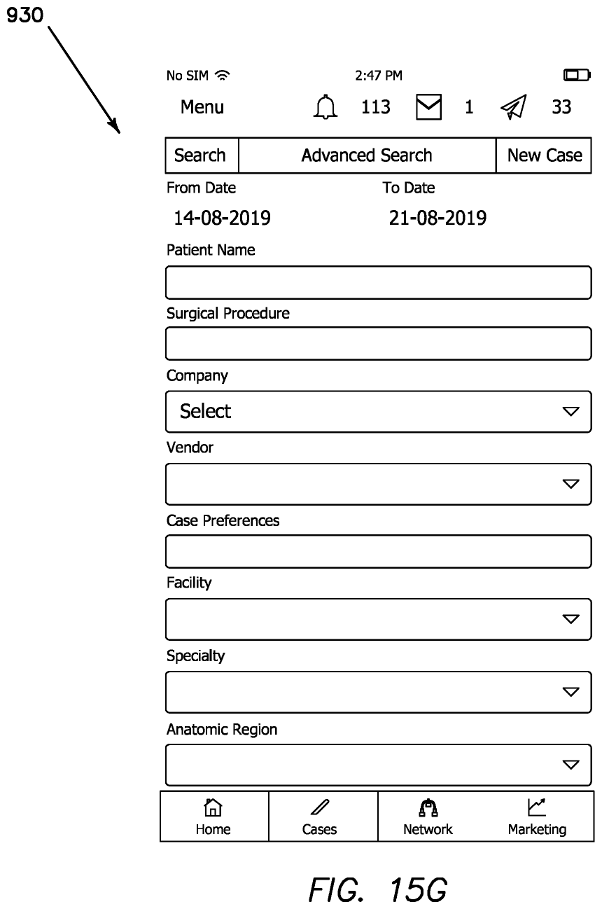

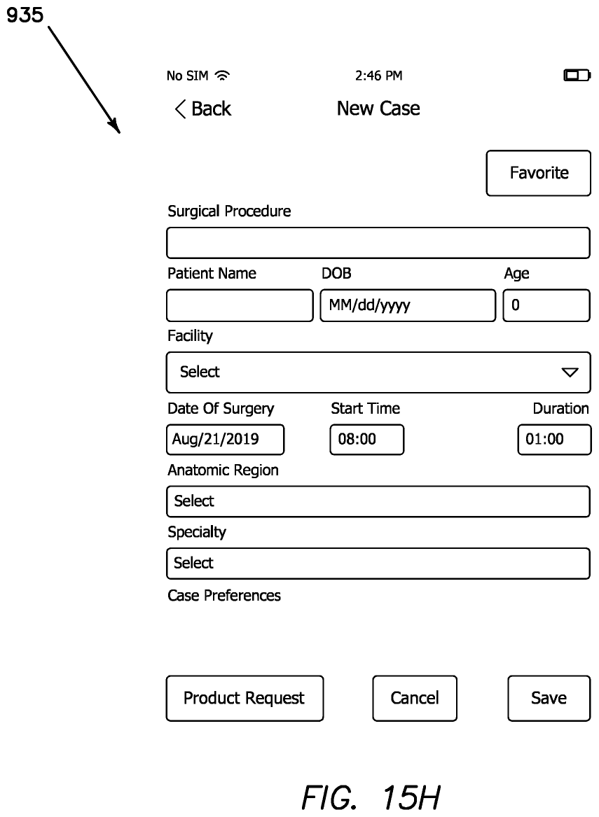

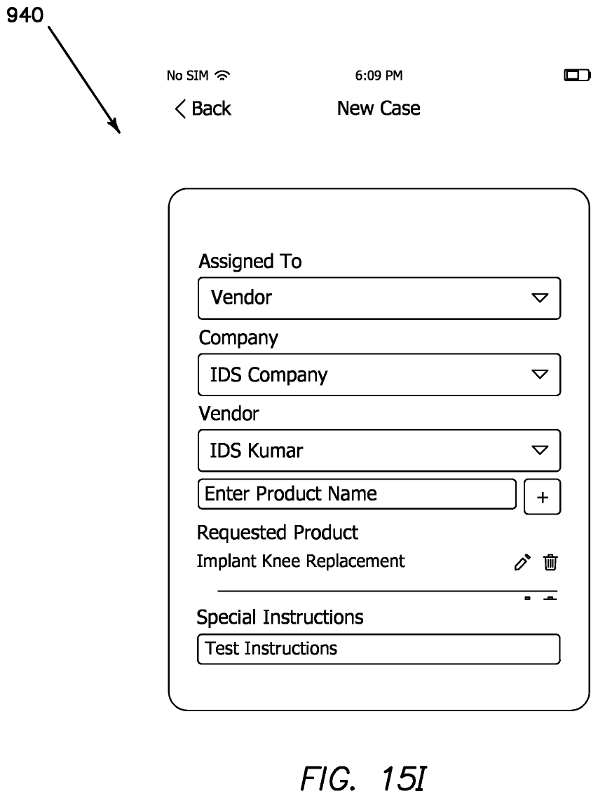

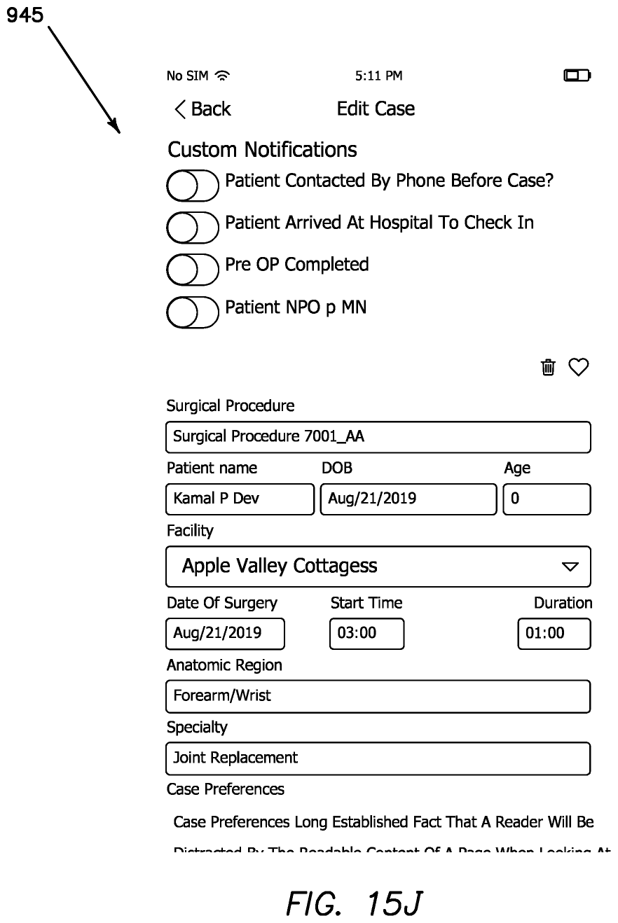

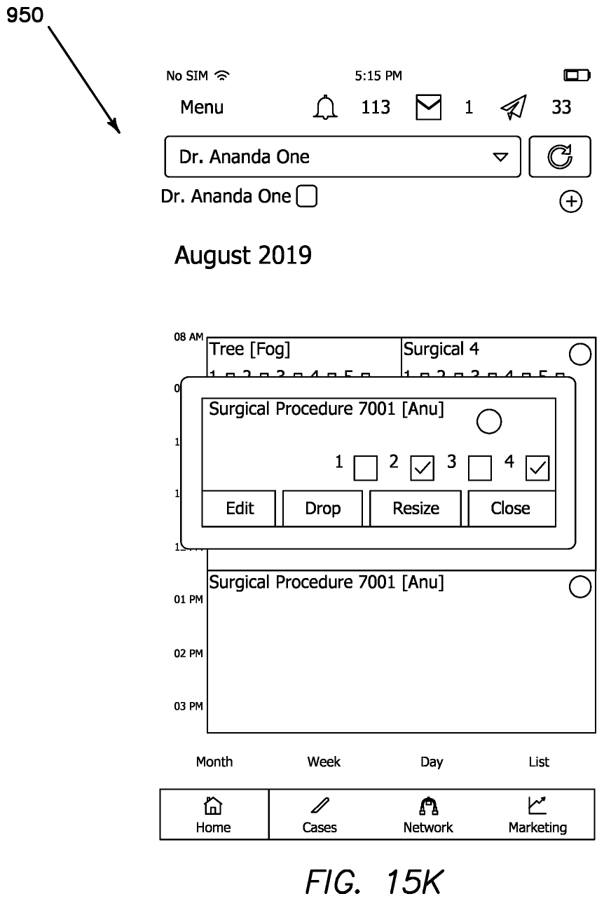

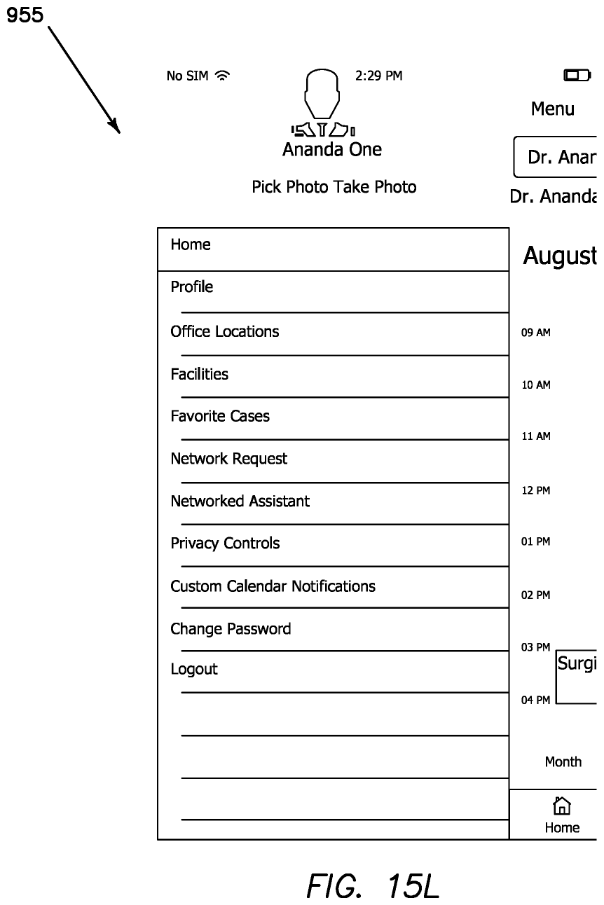

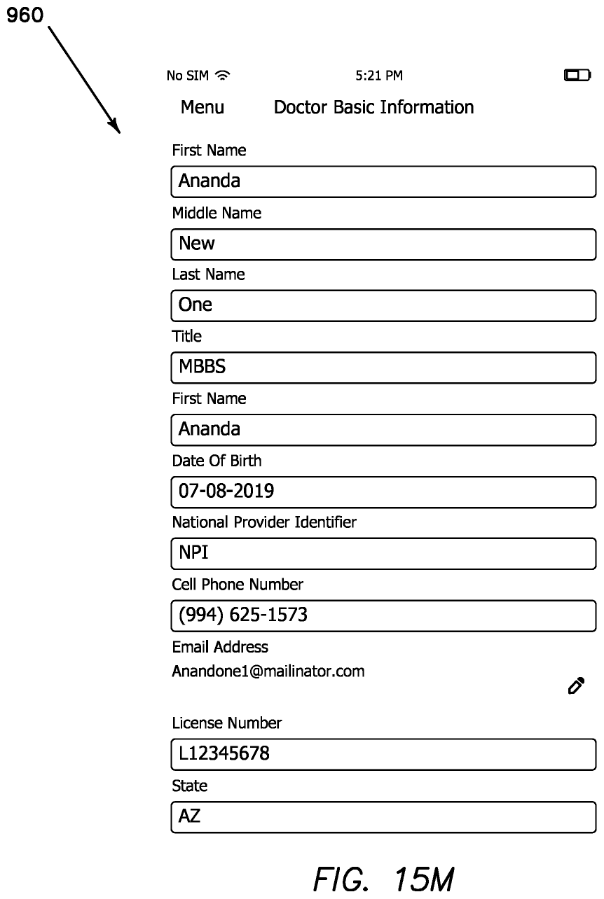

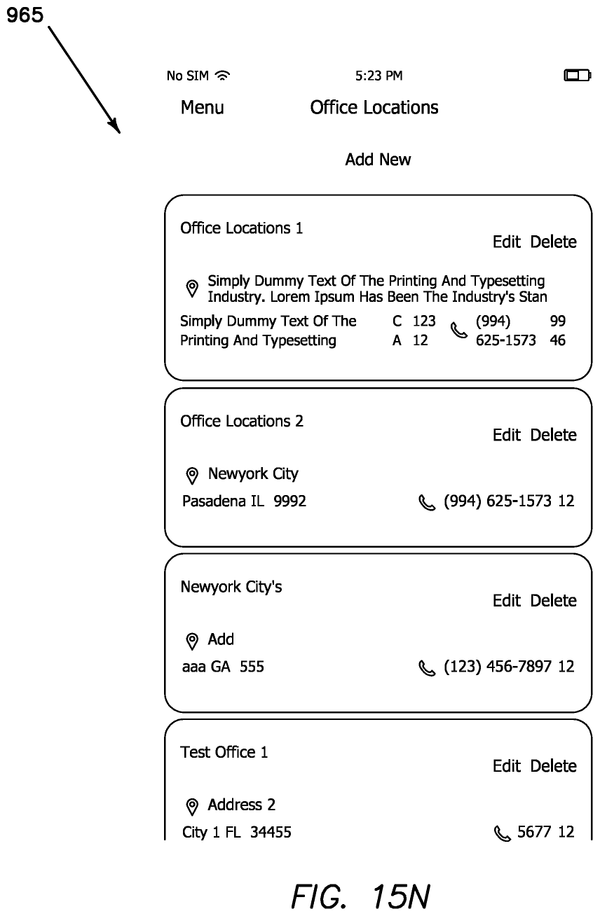

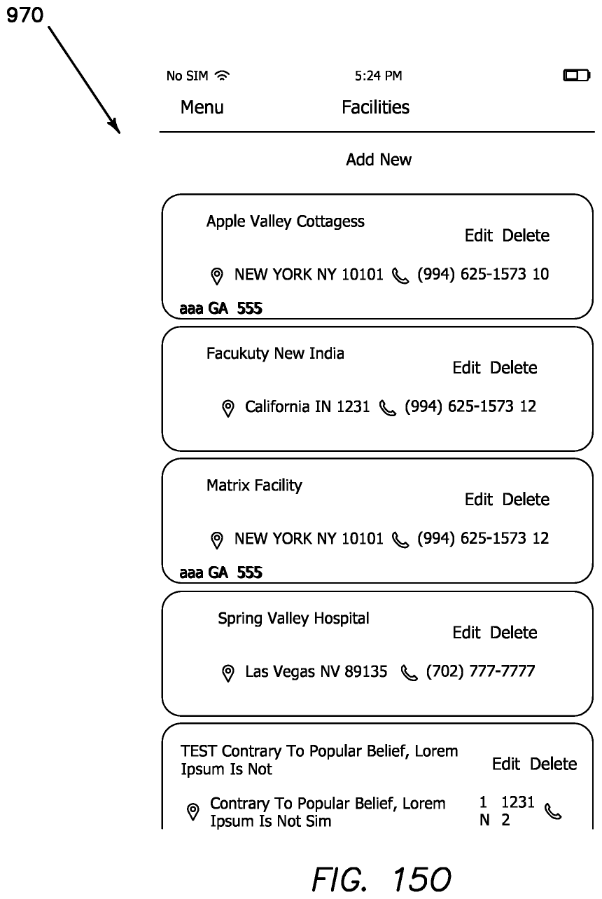

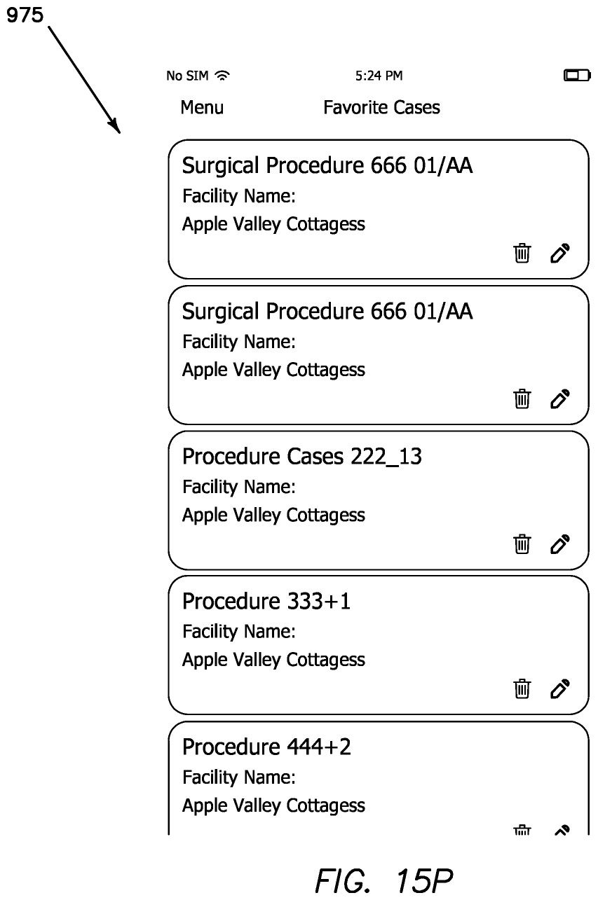

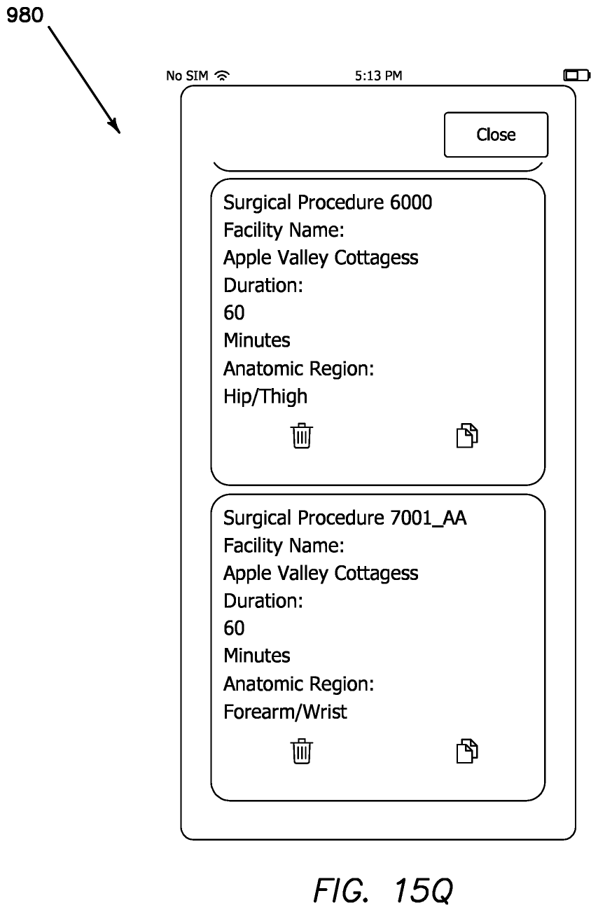

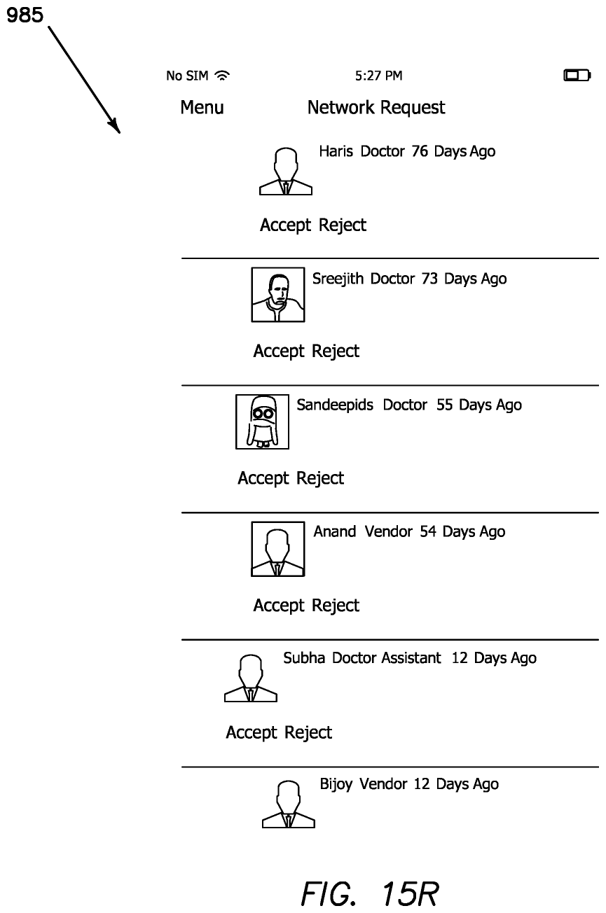

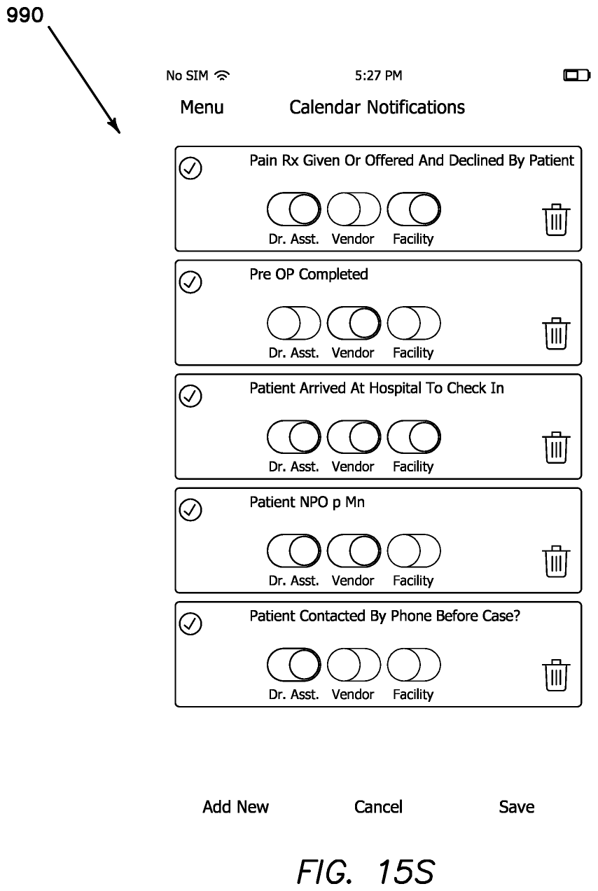

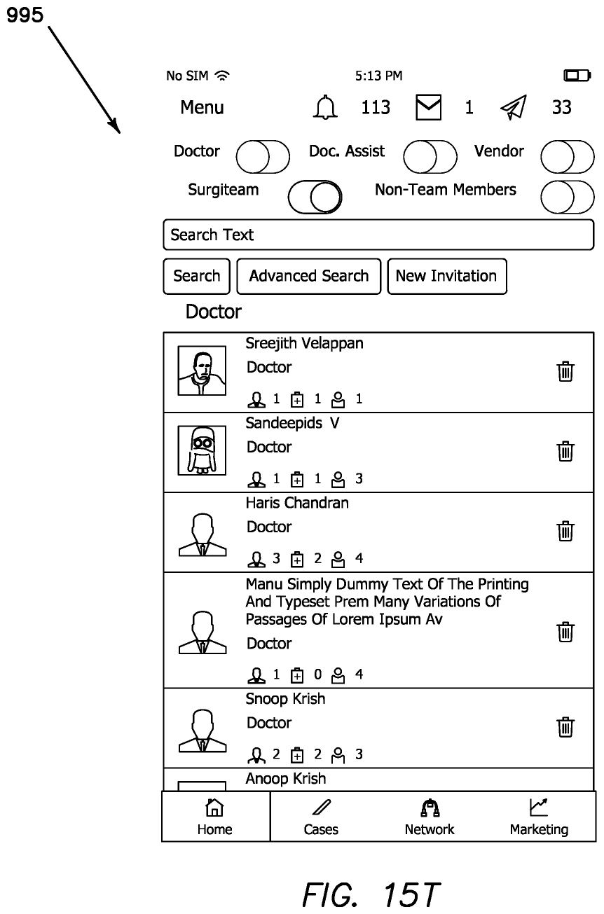

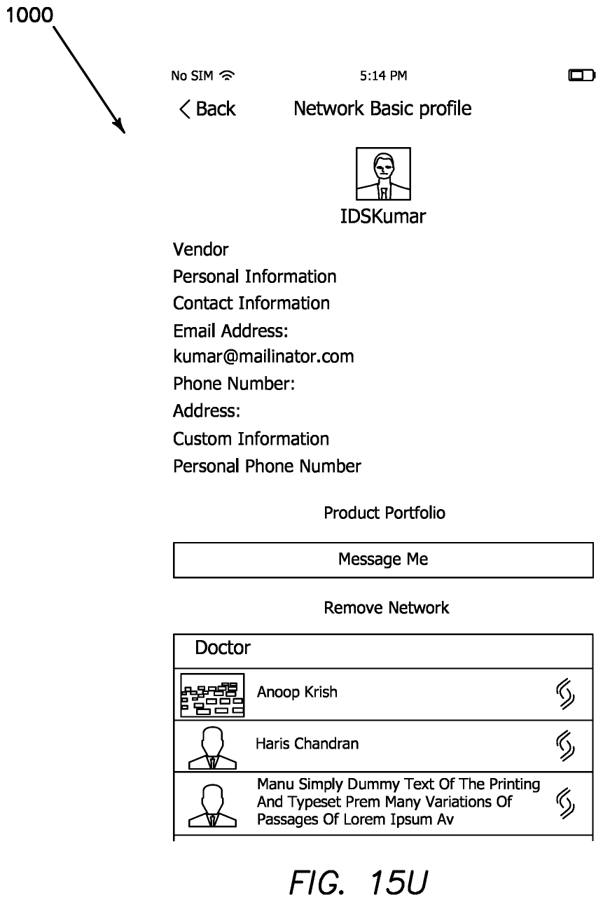

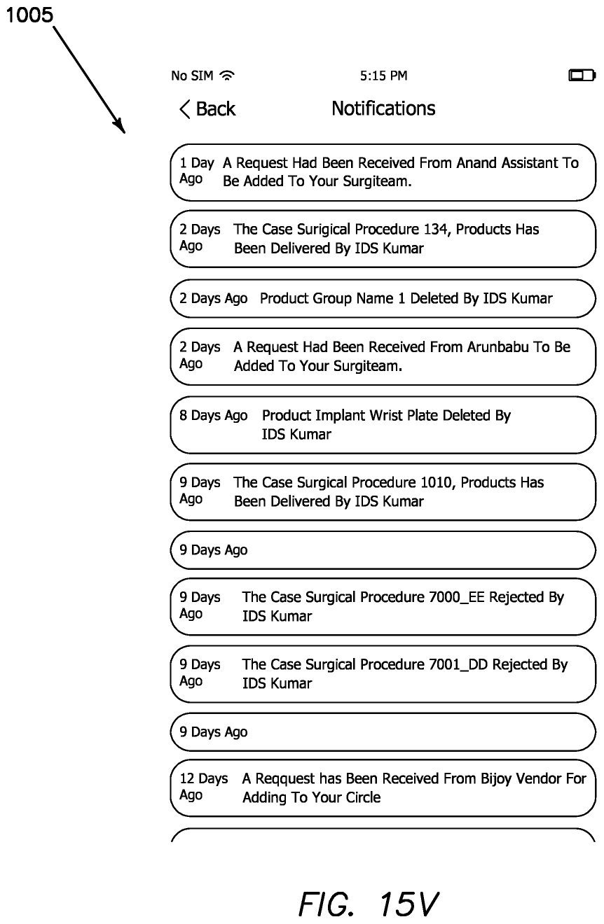

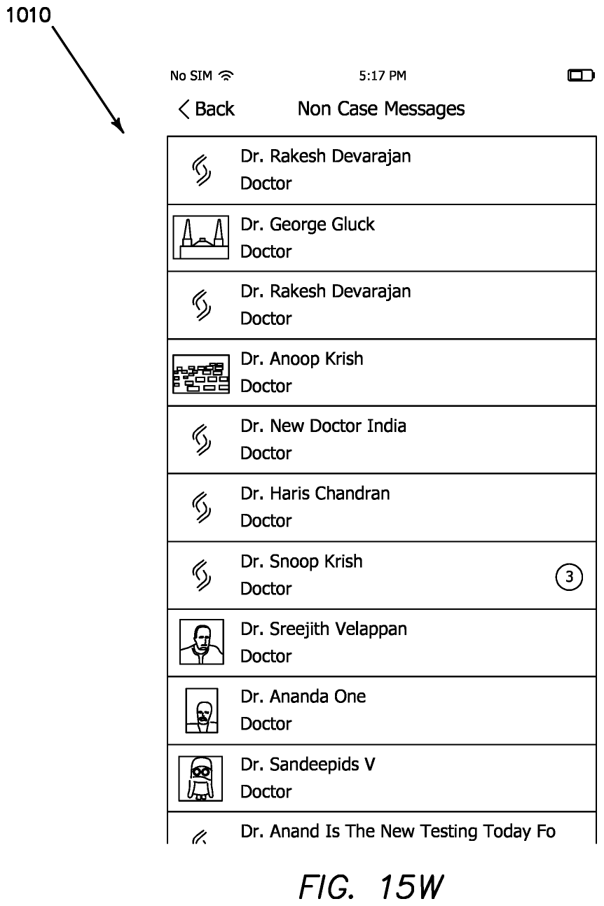

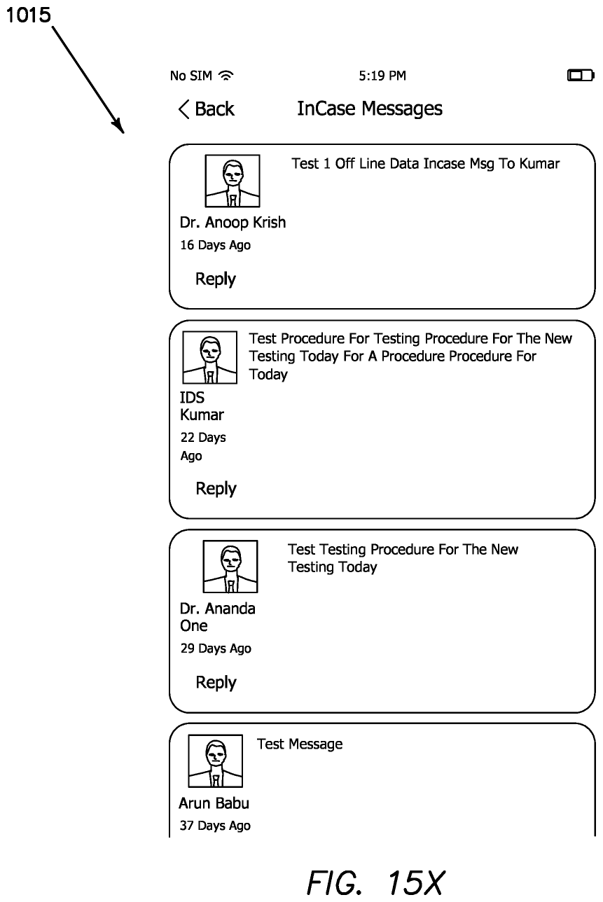

FIGS. 15A through 15X show exemplary screen shots as listed in Table 1 below.

TABLE 1

| FIG. | SCREEN SHOT |
|---|---|
| 15A | Login Page 900 |
| 15B | Calendar Day View 905 |
| 15C | Calendar List View 910 |
| 15D | Calendar Week View 915 |
| 15E | Calendar Month View 920 |
| 15F | Case Listing 925 |
| 15G | Advanced Search Page 930 |
| 15H | New Case Entry Page 935 |
| 15I | Product Request Page 940 |
| 15J | Edit Case Window 945 |
| 15K | Case Edit Dashboard 950 |
| 15L | Profile Menu 955 |
| 15M | Doctor Profile Page 960 |
| 15N | Office Locations 965 |
| 15O | Facilities 970 |
| 15P | Favorite Cases 975 |
| 15Q | Favorite Cases Cont. 980 |
| 15R | Network Request 985 |
| 15S | Calendar Notifications 990 |
| 15T | Network 995 |
| 15U | Network Basic Profile 1000 |
| 15V | Notifications 1005 |
| 15W | Non-Case Messages 1010 |
| 15X | Case Messages 1015 |

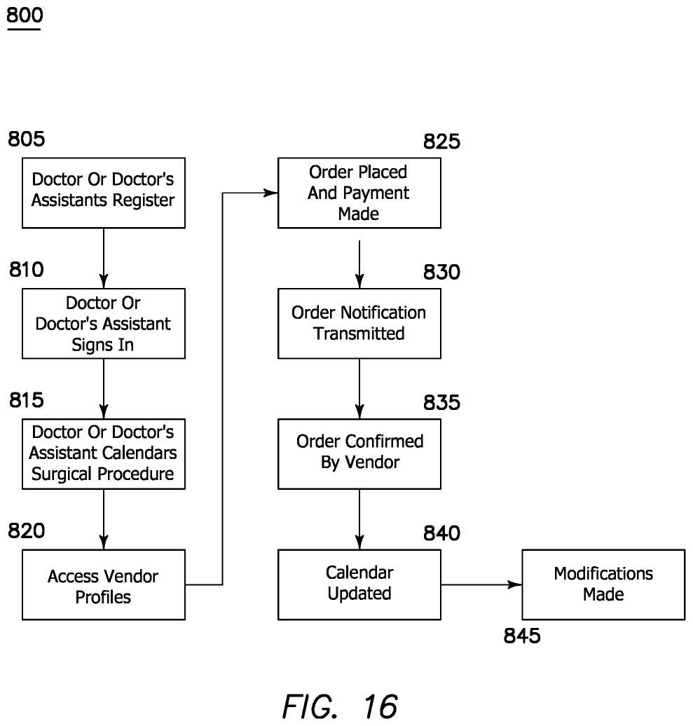

FIG. 16 shows a flow chart 800 detailing a method of utilizing the system by a doctor or doctor's assistant herein according to the embodiments of the present invention. At 805, the doctor and/or doctor's assistant register to use the web-based or app-based system. At 810, the doctor or doctor's assistant signs in to the web-based or app-based system. At 815, the doctor or doctor's assistant enters a surgical procedure on a given date and time on the dynamic calendar via the dashboard interface 600. At 820, the doctor or doctor's assistant accesses the vendor profiles via the web-based or app-based system. This access may be directed to a specific vendor, based on a pre-existing relationship, or a search for a new vendor distributing the implants and/or equipment needed for the calendared surgical procedure. At 825, the doctor or doctor's assistant places the order for the calendared surgical procedure and payment is made. Payment may be made using a credit card, digital wallet, dedicated account or other digital means. As set forth above, the order may include related information including (i) body part to be operated on; (ii) surgical procedure; (iii) patient details; (iv) date and time of surgery; (v) surgery sequence number and (vi) date and time the order needs to be delivered. At 830, the vendor, doctor and doctor's assistant receive notification via SMS, push notice and/or email. At 835, the order is confirmed by the vendor or vendor/sales rep receiving the order. At 840, upon confirmation, the order information may be stored in system memory, and, based on the order information, the calendar entry for the surgical procedure is updated to reflect the order. In one embodiment, each calendar entry for a surgical procedure entered by the doctor or doctor's assistant includes a reference number or code which allows the calendar entry to be updated by any user having access to the reference number or code. At 845, any changes to the surgical procedure, implants and/or equipment may be entered by the doctor or doctor's assistant via the web-based or app-based system.

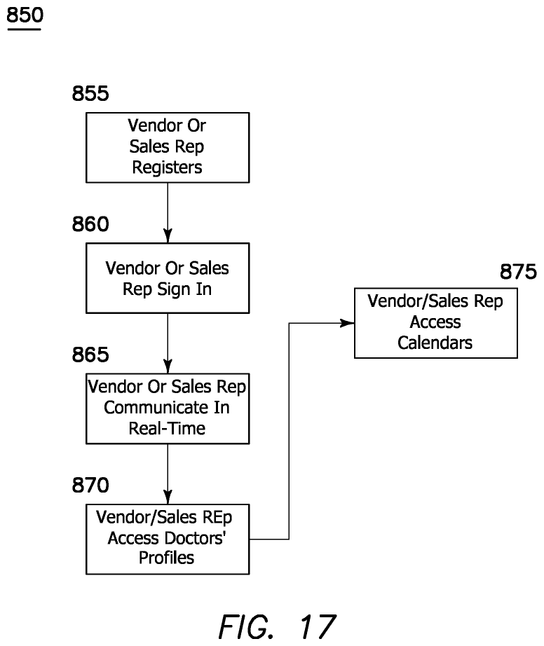

FIG. 17 shows a flow chart 850 detailing a method of utilizing the system by a vendor or vendor/sales rep herein according to the embodiments of the present invention. At 855, the vendor or vendor/sales rep register to use the web-based or app-based system. In one embodiment, the vendor may pay a subscription fee to advertise its products on the web-based or app-based system. At 860, the sales rep signs in to the web-based or app-based system. At 865, the sales rep may communicate in substantially real time with doctors, doctors' assistants and other users. The communications may be relative to an upcoming surgical procedure, a sales opportunity, a delivery status. At 870, vendors and sales rep may access doctor's profiles to seek sales leads. At 875, vendors and sales rep may access the calendar.

In one embodiment, the web-based or app-based system utilizes GPS to maintain a location of all interested parties the day of, or a pre-established time prior to, a surgical procedure. Via the system, the patient, surgeon and related surgical personnel may view the location of one another to ensure the surgical procedure will be starting on time. The location of the implant and/or equipment may also be tracked using a geolocation transmitter or similar article associated with the implant, equipment and/or packaging thereof to ensure the implant and/or equipment is on site.

In one embodiment, the web-based or app-based system permits doctors to rate vendors and their products and sales reps while also permitting vendors to rate doctors. The ratings may be public or private depending on the embodiment.

Although the invention has been described in detail with reference to several embodiments, additional variations and modifications exist within the scope and spirit of the invention as described and defined in the following claims.

We claim:

1. A computer-implemented system comprising:
memory and a hardware processor configured to:
generate a dynamic, digital community calendar accessible by at least registered surgeons and one or more registered implant vendors via one or more computer devices;
provide bi-directional electronic communications between said registered surgeons and said one or more registered implant vendors;
provide said surgeons with detailed digital information regarding implant inventories of said one or more registered implant vendors;
provide said surgeons with a digital order interface configured to allow surgeons to order implants from said one or more implant vendors, said order including a time, date and location for delivery of said order;
provide said one or more vendors with a digital acceptance interface configured to allow said one or more vendors to accept said order;
provide real-time status of delivery of said order; and
facilitate payment for said order.

2. The computer-implemented system of claim 1 wherein said dynamic, digital community calendar is further accessible by surgical patients.

3. The computer-implemented system of claim 1 further comprising GPS to track a location of said registered surgeons and/or ordered implants.

4. The computer-implemented system of claim 2 further comprising GPS to track a location of said patients.

5. The computer-implemented system of claim 1 further comprising a substantially real-time communication window.

6. The computer-implemented system of claim 1 wherein said memory and a hardware processor are further configured to: automatically update calendar entries responsive to said one or more vendors accepting said order.

7. The computer-implemented system of claim 1 wherein said memory and a hardware processor are further configured to: permit the entry of ratings between doctors, vendors and sales reps.

8. A tangible non-transitory machine readable medium storing instructions that, when executed, cause the computing device to perform a method, the method comprising:
generating a dynamic, digital community calendar accessible by at least registered surgeons and one or more registered implant vendors via one or more computer devices;
providing bi-directional electronic communications between said registered surgeons and said one or more registered implant vendors;
providing said surgeons with detailed digital information regarding implant inventories of said one or more registered implant vendors;
providing said surgeons with a digital order interface configured to allow surgeons to order implants from said one or more vendors, said order including a time, date and location for delivery of said order;
providing said one or more vendors with a digital acceptance interface configured to allow said one or more vendors to accept said order;
providing real-time status of delivery of said order; and
facilitating payment for said order.

9. The tangible non-transitory machine readable medium of claim 8, the method further comprising generating a dynamic, digital community calendar accessible by surgical patients.

10. The tangible non-transitory machine readable medium of claim 8, the method further comprising tracking a location of said registered surgeons and/or ordered implants.

11. The tangible non-transitory machine readable medium of claim 9, the method further comprising track a location of said patients.

12. The tangible non-transitory machine readable medium of claim 8, the method further comprising automatically updating calendar entries responsive to said one or more vendors accepting said order.

13. The tangible non-transitory machine readable medium of claim 8, the method further comprising: permitting entry of ratings between doctors, vendors and sales reps.

14. A computer-implemented system comprising:
memory and a hardware processor configured to:
generate a dynamic, digital community calendar accessible by at least registered surgeons and one or more registered implant vendors via one or more computer devices;
facilitate substantially real-time digital communications between said registered surgeons and said one or more registered implant vendors;
provide said surgeons with detailed digital information regarding implant inventories of said one or more registered implant vendors;
provide said surgeons with a digital order interface configured to allow surgeons to order implants from said one or more implant vendors, said order including a time, date and location for delivery of said order;
provide said one or more vendors with a digital acceptance interface configured to allow said one or more vendors to accept said order;
provide notifications of said order to a subject surgeon and subject implant vendor;
provide real-time status of delivery of said order; and
facilitate payment for said order.

15. The computer-implemented system of claim 14 wherein said dynamic, digital community calendar is further accessible by surgical patients.

16. The computer-implemented system of claim 14 further comprising GPS to track a location of said registered surgeons and/or ordered implants.

17. The computer-implemented system of claim 15 further comprising GPS to track a location of said patients.

18. The computer-implemented system of claim 14 wherein said memory and a hardware processor are further configured to: automatically update calendar entries responsive to said one or more vendors accepting said order.

19. The computer-implemented system of claim 14 wherein said memory and a hardware processor are further configured to: permit the entry of ratings between doctors, vendors and sales reps.
